(19) United States
(12) Patent Application Publication
(10) Pub. No.: US 2005/0026896 A1
(43) Pub. Date: Feb. 3, 2005

(54) Platinum(II) and platinum(IV) complexes and their use

(75) Inventor: Bernhard Keppler, Hockenheim (DE)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(73) Assignee: Faustus Forschungs Cie. Translational Cancer Research GmbH

(21) Appl. No.: 10/786,924

(22) Filed: Feb. 24, 2004

Related U.S. Application Data
(63) Continuation of application No. PCT/EP02/09471, filed on Aug. 23, 2002.

(30) Foreign Application Priority Data
Aug. 24, 2001 (DE) 101 41 528.1

(51) Int. Cl. A61K 31/555; C07F 15/00; A61K 31/28

(52) U.S. Cl. 514/184; 514/492; 546/2; 548/402; 556/137

(57) ABSTRACT

Platinum(II) complexes are provided selected from the group consisting of compounds of the general formulae I to IV and physiologically acceptable addition salts thereof, 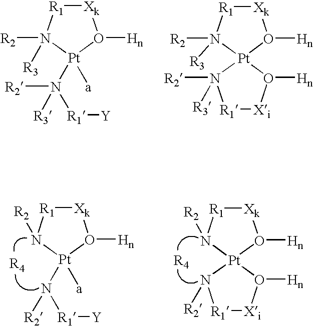
wherein the radicals R¹ to R⁴, R^{1′} to R^{3′}, X, X′, Y, a, i, k and n are defined as in the description, and mixtures of the compounds for use as prophylactic and/or therapeutic agents for the treatment of diseases, and cytostatic platinum complexes with the general formulae
   [Pt^{II}(NH₃)ₙ(A)ₙ(Z)₂₋ₙ] or [Pt^{IV}(NH₃)ₙ(A)ₙ(Z)₂₋ₙX₂]and
   [Pt^{II}(A)₁(Z)₂] or [Pt^{IV}(A)₁(Z)₂X₂] wherein A, Z and n are as defined in the description.

### CROSS-REFERENCE TO RELATED APPLICATIONS

This application is a continuation of International Application No. PCT/EP02/09471, filed Aug. 23, 2002, the disclosure of which is incorporated herein by reference.

### BACKGROUND OF THE INVENTION

Platinum compounds are used as complexes with organic ligands in the therapy of tumors, in particular of ovarian and colorectal carcinoma. The prerequisite for the cytostatic effectiveness of this type of platinum compound is, as generally accepted, the reaction of the platinum or the activated platinum with DNA or other biomolecules. This reaction can be reproduced experimentally in vitro (Zenker, A.; Galanski, M.; Bereuter, T. L.; Keppler, B. K.; Lindner, W.; J. Biol. Inorg. Chem., 5(4): 498-504 (2000). Currently, the compounds cis-platinum, carboplatinum and oxali-platinum are of special clinical significance. These substances and analogous compounds are in part associated with substantial toxic side effects, which in particular affect the kidneys, the auditory organ, other nervous organs including the eye and the bone marrow. These side effects can limit the dose and consequently impair the success of treatment.

The previously used compounds consist of platinum(II) and platinum(IV) complexes with two coordinating groups containing nitrogen and two or more anionic coordinating groups, which can be linked together chemically in each case (DE 4,041,353 Keppler, B. K., EP 0,367,974 Kolak, C. et al., EP 0,167,071 Kolak, C. et al., U.S. Pat. No. 5,434,256 Kokhar, A. & Siddik, Z. H., U.S. Pat. No. 4,607,114 Nakayama, Y. et al., U.S. Pat. No. 4,704,464 Brunner, H. et al.).

Platinum compounds, which carry two ethanol amine ligands and are coordinated to the platinum both via the nitrogen and by the oxygen, have previously been described as inactive (Kuroda et al., 2000). Generally, no pronounced cytotoxic activity is known for this type of closed-ring hydroxyalkyl amines.

Uckun et al. (WO 01/36431 A1) describe in their patent application only the corresponding open-ring compounds, i.e. platinum complexes with hydroxyalkyl amine ligands, which however are only coordinated to the platinum via the nitrogen. Also, no special effectiveness has been described for this type of compound.

### BRIEF SUMMARY OF THE INVENTION

The object of the invention is to provide cytostatic platinum compounds for medicaments with an improved therapeutic index, and therefore fewer side effects for the same effectiveness or improved effectiveness without increasing the side effects.

The object is solved by a platinum(II) complex selected from the group consisting of compounds of the general formulae I to IV and physiologically acceptable addition salts of them,

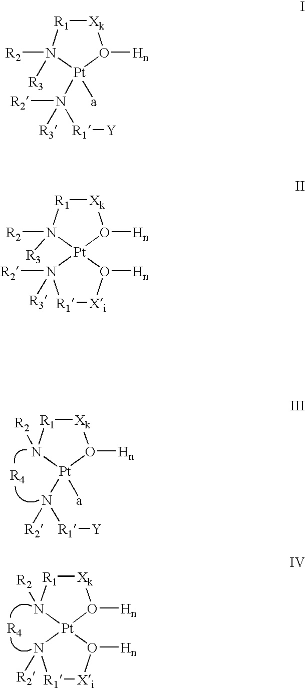

wherein the radicals

R¹ and R^{1′} are selected independently of one another from the group consisting of substituted or unsubstituted alkylene and alkenylene radicals, which can be substituted by halogen, alkyl, cycloalkyl, hydroxy, carboxy, sulphate, phosphate and/or heterocyclic compounds,

R₂, R³, R^{2′}, R^{3′} are selected independently of one another from the group consisting of —(CH₂)ₘ—OH, —H, substituted or unsubstituted alkyl radicals, saturated and unsaturated cyclic radicals and heterocyclic compounds which can be substituted by halogen, hydroxy, carboxy, sulphate and/or phosphate,

m signifies a natural number from 2 to 5,

R⁴ is selected from the group consisting of substituted or unsubstituted alkylene, alkenylene, cycloalkylene and cycloalkenylene radicals, and aromatic and heterocyclic radicals,

X, X′=—S(O₂)—,

k, i=0 or 1,

Y=—OH, —SO₃H,

n=0 or 1, and

a is selected from the group consisting of halogenides, OH⁻, OH₂, carboxylate, sulphate and sulphonate

and mixtures of the compounds for application as prophylactic and/or therapeutic agents in the treatment of diseases.

Furthermore, the object is solved by a platinum(IV) complex with a single or double intramolecular cyclization selected from the group consisting of compounds of the general formulae Ia to IVa and physiologically compatible addition salts of them,

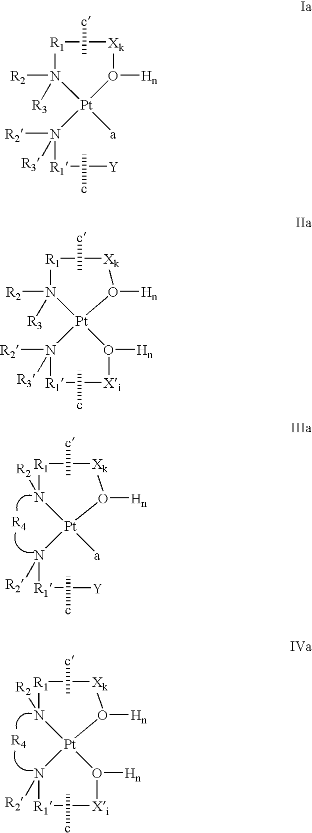

wherein the radicals

R¹ and R^{1′} are selected independently of one another from the group consisting of substituted or unsubstituted alkylene and alkenylene radicals, which can be substituted by halogen, alkyl, cycloalkyl, hydroxy, carboxy, sulphate, phosphate and/or heterocyclic compounds,

R², R³, R^{2′}, R^{3′} are selected independently of one another from the group consisting of —(CH₂)ₘ—OH, —H, substituted or unsubstituted alkyl radicals, saturated and unsaturated cyclic radicals or heterocyclic compounds which can be substituted by halogen, hydroxy, carboxy, sulphate and/or phosphate,

m signifies a natural number from 2 to 5,

R⁴ is selected from the group consisting of substituted or unsubstituted alkylene, alkenylene, cycloalkylene and cycloalkenylene radicals, and aromatic and heterocyclic radicals,

X, X′=—S(O₂)—,

k, i=0 or 1,

Y=—OH, —SO₃H,

n=0 or 1, and

a, c, c′ are selected independently of one another from the group consisting of halogenides, OH⁻, OH₂, carboxylate, sulphate and sulphonate

and mixtures of the compounds for use as prophylactic and/or therapeutic agents for the treatment of diseases.

Preferably, R¹ and R^{1′} are selected independently of one another from the group consisting of substituted or unsubstituted C₁₋₆-alkylene and C₂₋₆-alkenylene radicals, which can be substituted by halogen, alkyl, alkenyl, cycloalkyl, cycloalkenyl, aryl, hydroxy, carboxy, sulphate, phosphate and/or heterocyclic compounds. Furthermore, in the platinum(II) complex or platinum(IV) complex according to the invention R¹ and R^{1′} are preferably selected independently of one another from the group consisting of C₂₋₅-alkylene radicals and C₂₋₅-alkenylene radicals.

Preferably, R², R³, R^{2′}, R^{3′} are selected independently of one another from the group consisting of —(CH₂)ₘ—OH, —H, substituted or unsubstituted C₁₋₆-alkyl radicals, saturated and unsaturated cyclic C₃₋₆ radicals or heterocyclic compounds which can be substituted by halogen, hydroxy, carboxy, sulphate and/or phosphate. Particularly preferred, R², R³, R^{2′}, R^{3′} are selected independently of one another from the group consisting of hydrogen, methyl and ethyl radicals.

Preferably, the radical R⁴ is selected from the group consisting of substituted or unsubstituted C₁₋₆-alkylene, C₂₋₆-alkenylene, C₃₋₆-cycloalkylene and C₃₋₆-cycloalkenylene radicals and aromatic and heterocyclic C₃₋₆ radicals. In particular, the radical R⁴ is preferably an ethylene radical.

In a further preferred embodiment of the platinum(II) complex or platinum(IV) complex according to the invention, the radicals

R¹ , R¹=re substituted or non-substituted alkylene, preferably C₁₋₆-alkylene,

k, i=0, and/or

Y=—OH.

Furthermore, the radicals in the platinum(II) complex or platinum(IV) complex according to the invention preferably have the following significance:

R¹, R^{1′}=ethylene

k, i=0,

Y=—OH, and/or

n=0.

The radicals a, c, c′ are preferably selected from halogenides, in particular chloride and OH⁻. Furthermore, if a, c and/or c′ are OH₂, then the platinum(II) or platinum(IV) complexes according to the invention have in each case single, double or triple positive charges, depending on the number of OH₂ radicals present. In this case a required number of anions is present to balance the positive charge, preferably selected from the group consisting of halogenides, in particular chloride, nitrate, sulphate, phosphate, HCO₃, RCOO, with R═C₁₋₆-alkyl, C₂₋₆-alkenyl, C₃₋₆-cycloalkyl, C₃₋₆-cycloalkenyl or aryl.

If n is equal to one, then the platinum(II) or platinum(IV) complexes according to the invention have in each case, depending on the number of H atoms present, single (complexes of the formulae I, III, Ia and IIIa) or single or double (complexes of the formulae II, IV, Ia and IVa) positive charges. In this case a required number of anions is present to balance the positive charge, preferably selected from the group consisting of halogenides, in particular chloride, nitrate, sulphate, phosphate, HCO₃, RCOO, with R═C₁₋₆-alkyl, C₂₋₆-alkenyl, C₃₋₆-cycloalkyl, C₃₋₆-cycloalkenyl or aryl.

In preferred embodiments the platinum(II) complex is a compound of the general formula III, more preferably a compound of the general formula I and especially preferred is a compound of the general formula II.

In preferred embodiments the platinum(IV) complex is a compound of the general formula IIIa, more preferably a compound of the general formula Ia and especially preferred is a compound of the general formula IIa.

The above described compounds of the general formulae I to IV and Ia to IVa can be used for the manufacture of a prophylactic and/or therapeutic agent for the treatment of tumor diseases.

The object of this invention is also solved by platinum(II) or platinum(IV) compounds according to the general formula (V),
[Pt^{II}(NH₃)ₙ(A)ₙ(Z)₂₋ₙ] or [Pt^{IV}(NH₃)ₙ(A)₁(Z)₂₋ₙX₂] (V)

wherein

n is equal to 0 or 1,

A, X are selected independently of one another from the group consisting of halogenides, OH⁻, OH₂, carboxylate, sulphate and sulphonate, and

Z is selected from the group consisting of hydroxyalkyl amine, hydroxyalkenyl amine, sulphoalkyl amine and sulphoalkenyl amine, which is substituted at at least one of the CH₂— or CH groups by a halogen, alkyl, alkenyl, cycloalkyl, cycloalkenyl, aryl, hydroxyl, carboxyl, sulphate, phosphate radical and/or a heterocyclic compound and additionally the amino nitrogen is substituted with these radicals, wherein
for n equal to 0, the two radicals Z present in the molecule can be linked via a radical selected from the group consisting of substituted or unsubstituted alkylene, alkenylene, cycloalkylene and cycloalkenylene radicals and a heterocyclic compound, preferably ethane-1,2-diyl.

Furthermore, the object of this invention is solved by platinum(II) or platinum(IV) compounds according to the general formula (VI),
[Pt^{II}(A)₁(Z)₂] or [Pt^{IV}(A)₁(Z)₂X₂] (VI)

wherein

A and X are selected independently of one another from the group consisting of halogenides, OH⁻, OH₂, carboxylate, sulphate and sulphonate, and

Z is selected from the group consisting of hydroxyalkyl amine, hydroxyalkenyl amine, carboxyalkyl amine, carboxyalkenyl amine, sulphoalkyl amine and sulphoalkenyl amine, which is substituted at at least one of the CH₂— or CH groups by a halogen, alkyl, alkenyl, cycloalkyl, cycloalkenyl, aryl, hydroxyl, carboxyl, sulphate, phosphate radical and/or a heterocyclic compound and additionally the amino nitrogen can be substituted by these radicals, wherein
the two Z radicals present in the molecule can be linked via a radical selected from the group consisting of alkylene, alkenylene, cycloalkylene and cycloalkenylene radicals and a heterocyclic compound, or substituted alkylene, alkenylene, cycloalkylene and cycloalkenylene.

In a preferred embodiment two Z radicals present in the molecule can be linked via ethylene.

If the platinum(II) or platinum(IV) complexes of the formulae (V) or (VI) according to the invention have one or more positive charges due to the substitutions, then the same applies as stated with reference to the platinum(II) or platinum(IV) complexes of the formulae (I) to (IVa) according to the invention and the anions are preferably selected as shown above.

Preferably, organic or inorganic addition salts of the platinum(II) and platinum(IV) complexes according to the invention can be formed with the following anions: chloride, bromide, phosphate, carbonate, nitrate, perchlorate, sulphate, citrate, lactate, tartrate, maleate, fumarate, mandelate, benzoate, ascorbate, cinnamate, glycolate, methane sulphonate, formiate, malonate, naphthalin-2-sulphonate, salicylate and/or acetate. H⁺, sodium and/or potassium cations can be used as possible cations.

Furthermore, the object of this invention is solved by a platinum(II) or platinum(IV) compound of the general formula (V) or (VI) for use as a prophylactic and/or therapeutic agent for the treatment of diseases.

The platinum(II) or platinum(IV) compound of the general formula (V) or (VI) can furthermore be used for the manufacture of a prophylactic and/or therapeutic agent for the treatment of tumor diseases.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is an X-ray structural analysis of a compound of Example 4;

FIG. 2 shows plots of the reactivity of the compound of Example 8 under physiological and patho-physiological conditions; and

FIG. 3 is a plot of the reactivity of the compound of Example 9 under patho-physiological conditions.

### DETAILED DESCRIPTION OF THE INVENTION

The platinum(II) or platinum(IV) complexes are stabilized platinum compounds consisting of complexes in which at least one of the ligands chelates the platinum via an N as well as O and can simultaneously replace a counterion, classically chloride. Consequently, the reactivity of the compound to biomolecules (in particular DNA) is many times reduced compared to the open-ring compound. Surprisingly however, a strong reactivity, comparable to that of cis-platinum, could be observed for these compounds under the patho-physiologically relevant conditions. Previously, these compounds were regarded as inactive (Kuroda et al., 1983).

Due to the described structures an increased selectivity of cytotoxic platinum compounds for tumors and therefore an improved therapeutic index can be achieved. The compounds according to the invention also exhibit an increased selectivity for solid tumors.

In the following, examples of platinum(II) complexes of the general formulae I-IV are described:

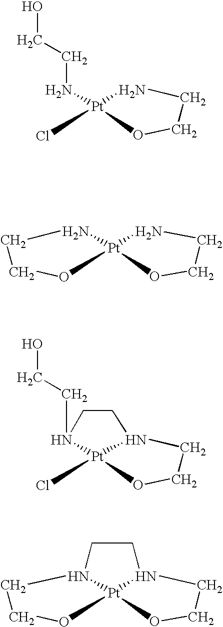

In the following, examples of platinum(IV) complexes of the general formulae Ia-IVa are described:

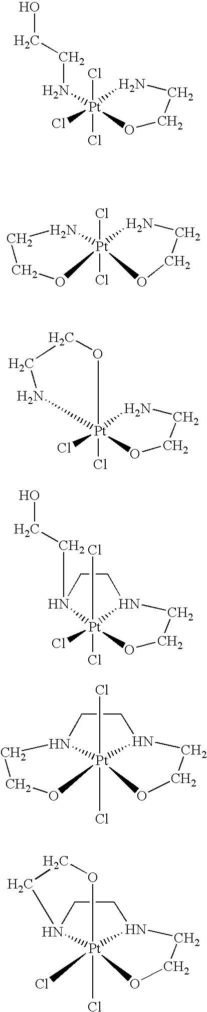

Platinum(II) complexes of the general formulae I-IV or platinum(IV) complexes of the general formulae Ia-IVa, wherein the radicals R¹ and R^{1′} signify ethylene, k is equal to 0 and Y is —OH, are preferred, in particular however platinum(II) complexes or platinum(IV) complexes of the general formulae I-IV resp. Ia-IVa are preferred, wherein the radicals R¹ and R^{1′} signify ethylene and k and i are equal to 0.

In a further aspect, this invention relates to the use of platinum(II) complexes of the general formulae Ib to IVb in the manufacture of medicaments for the therapy of tumor diseases.

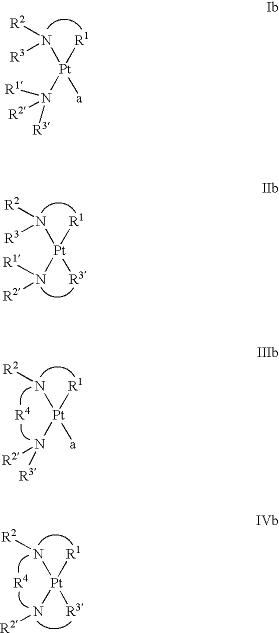

wherein the radicals R¹, R^{1′}, R², R^{2′}, R³, R^{3′} and a in the formulae Ib to IVb have the following meanings:

R¹ and R^{3′} are selected from the group of hydroxyalkyls and hydroxyalkenyls and originate from hydroxyalkyls and hydroxyalkenyls substituted with halogens, alkyls, cycloalkyls, heterocyclic compounds or functional groups such as hydroxy, carboxy, sulphate or phosphate, wherein the hydroxyalkyls and hydroxyalkenyls can be present protonated or deprotonated,

R², R³, R^{1′}, R^{2′} are selected from the group —CH₂—CH₂—OH, —CH₂—CH₂—CH₂—OH, —CH₂—CH₂—CH₂—CH₂—OH, —CH₂—CH₂—CH₂—CH₂—CH₂—OH, —H, methyl, ethyl, saturated or unsaturated cyclic radicals, also heterocyclic compounds, as well as their halogen, hydroxy, carboxy, sulphate or phosphate derivatives,

R⁴ is selected from alkyl, alkylene, cycloalkyl, cycloalkene, heterocyclic radicals or substituted alkyls and alkylenes, cycloalkyl and cycloalkene, but preferably the radical R⁴ can be ethane-1,2-diyl,

and a belongs to the group of halogenides (fluorine, chlorine, bromine, iodine), OH⁻, OH₂, carboxylate, sulphate or sulphonate.

R¹ and R^{3′} may preferably signify carboxy alkyls or carboxy alkenyls as well as carboxy alkyls and carboxy alkenyls substituted with halogens, alkyls, cycloalkyls, heterocyclic compounds or functional groups such as hydroxy, carboxy, sulphate or phosphate and the carboxy alkyls or carboxy alkenyls can be present protonated or deprotonated.

R¹ and R^{3′} may also preferably signify sulphoalkyls or sulphoalkenyls as well as sulphoalkyls and sulphoalkenyls substituted with halogens, alkyls, cycloalkyls, heterocyclic compounds or functional groups such as hydroxy, carboxy, sulphate or phosphate and the sulphoalkyls or sulphoalkenyls can be present protonated or deprotonated.

R¹ and R^{3′} may also preferably signify —CH₂—CH₂—OH, —CH₂—CH₂—CH₂—OH, —CH₂—CH₂—CH₂—CH₂—OH, —CH₂—CH₂—CH₂—CH₂—CH₂—OH, —CH₂—COOH, —CH₂—CH₂—COOH, —CH₂—CH₂—CH₂—COOH, —CH₂—CH₂—SO₃H, —CH₂—CH₂—CH₂—SO₃H, —CH₂—CH₂—CH₂—CH₂—SO₃H and —CH₂—CH₂—CH₂—CH₂—CH₂—SO₃H and R¹ and R^{3′}=—CH₂—CH₂—OH, —CH₂—CH₂—CH₂—OH, —CH₂—CH₂—CH₂—CH₂—OH, —CH₂—CH₂—CH₂—CH₂—CH₂—OH, —CH₂—COOH, —CH₂—CH₂—COOH, —CH₂—CH₂—CH₂—COOH, —CH₂—CH₂—SO₃H, —CH₂—CH₂—CH₂—SO₃H, —CH₂—CH₂—CH₂—CH₂—SO₃H and —CH₂—CH₂—CH₂—CH₂—CH₂—SO₃H can be present protonated or deprotonated.

In a further aspect, this invention relates to the use of platinum(IV) complexes with single or double intramolecular cyclization of the general formulae Ic to IVc for the manufacture of medicaments for the therapy of tumor diseases,

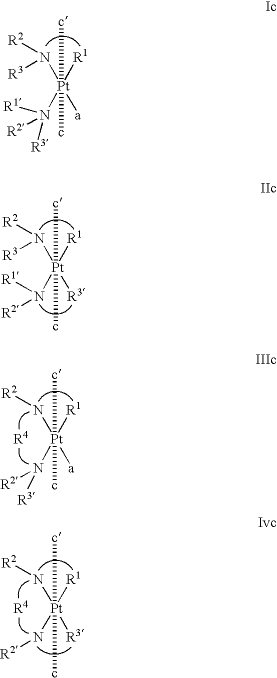

wherein the radicals in the formulae Ic to IVc are selected as described above with reference to the formulae Ib to IVb.

In a further aspect this invention relates to the use of platinum(II) and platinum(IV) complexes of the general formula V for the manufacture of medicaments for the therapy of tumor diseases,

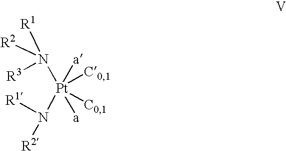

wherein the radicals are selected as described above with reference to the formulae Ib to IVb and a, a′ as well as c, c′ belong to the group of halogenides (fluorine, chlorine, bromine, iodine), OH⁻, OH₂, carboxylate, sulphate or sulphonate, wherein c is omitted for platinum(II) compounds (c₀).

Preferably, the radicals are selected as described above with reference to the formulae Ib to IVb.

In the following the prophylactic and/or therapeutic agent with at least one platinum(II) and/or platinum(IV) complex and/or compound for the treatment of diseases is explained in more detail.

The agent according to the invention is primarily administered intravenously, but also intramuscularly, intraperitoneally, subcutaneously or perorally. External or pulmonary application is also possible. Preferably, it is administered by intravenous injection or by intravenous infusion.

The agent is manufactured according to methods known per se, wherein the compound is used in combination with suitable pharmaceutical carrier substances. In this respect, the content of active substance in this mixture is normally 0.1 to 99.5, preferably 0.5 to 95% by weight of the total mixture.

The agent can be applied in any suitable formulation with the prerequisite that the establishment and maintenance of a sufficient level of active substance is ensured. This can, for example, be achieved by the peroral or parenteral administration in suitable doses. Advantageously, the pharmaceutical preparation of the active substance is provided in the form of standard doses which are matched to the desired administration. A standard dose can, for example, be a tablet, a coated tablet, capsule, suppository or a measured volume of a powder, granulate, solution, emulsion or suspension.

A “standard dose” for the purposes of this invention is taken to mean a physically determined unit which contains an individual quantity of the active constituent in combination with a pharmaceutical carrier substance and its content of active substance corresponds to a fraction or multiple of a therapeutic single dose. A single dose preferably contains the quantity of active substance which is administered during an application and which normally corresponds to a whole, half, third or quarter of the daily dose. If only a fraction, such as half or quarter of the standard dose is needed for a single therapeutically administered dose, then the standard dose is advantageously divisible, e.g. in the form of a tablet with a dividing groove.

The agent can, if the active substance is present in standard doses and is intended for applications, e.g. on persons, contain about 0.1 to 500 mg, preferably 10 to 200 mg and particularly 50 to 150 mg of active substance.

Generally in human medicine, the active substance(s) are administered in a daily dose of 0.1 to 5, preferably 1 to 3 mg/kg of body weight, where necessary in the form of a number, preferably 1 to 3, of single intakes for achieving the desired results. A single intake contains the active substance(s) in quantities of 0.1 to 5, preferably 1 to 3 mg/kg of body weight. With oral treatment similar dosages can be applied.

The therapeutic administration of the agent can occur 1 to 4 times daily at specified or varying time points, e.g. in each case before meals and/or in the evening. However, it may be necessary to deviate from the quoted dosages depending on the type, body weight and age of the individual to be treated, the type and severity of the disease, the type of preparation and the application of the agent as well as the time period or interval within which the administration occurs. Consequently, in some cases it may be sufficient to use less than the amount of active substance mentioned above, whereas in other cases the above listed quantity of active substance must be exceeded. It may also be practicable to administer the agent only once or at intervals of a number of days.

The specification of the necessary optimum dosage and type of application of the active substance can be made by any specialist based on his specialist knowledge.

The agent normally comprises at least an active substance and non-toxic, pharmaceutically acceptable agent carriers, which as additive or dilution agents, are employed, for example, in solid, semi-solid or liquid form or as a means of enclosure, for example in the form of a capsule, a tablet coating, a bag or another container for the therapeutically active constituent. A carrier material may, for example, act as an intermediary for the ingestion of the agent by the body, as a formulation agent, sweetener, taste modifier, colorant or as a preservative.

For peroral application, for example, tablets, coated tablets, capsules, for example of gelatin, dispersible powder, granulate, aqueous and oily suspensions, emulsions, solutions and syrups can be employed.

Tablets can contain inert filling agents, e.g. starches, lactose, microcrystalline cellulose, glucose, calcium carbonate, or sodium chloride; binding agents, e.g. starches, polyethylene glycols (PEGs), polyvinyl pyrrolidon (PVP), gelatin, cellulose derivatives, alginates or arabine; lubricating agents, e.g. magnesium stearate, glycerin monostearate, stearic acid, silicone oils or talcum; decomposition agents, e.g. starches, microcrystalline cellulose or cross-linked polyvinyl pyrrolidon; taste modifiers or colorants. They can also be provided with a coating which is produced such that it causes delayed release and resorption of the agent in the gastro-intestinal tract, so that, for example, improved compatibility, protraction or retardation is achieved.

Gelatin capsules may contain the pharmaceutical substance mixed with a solid, e.g. lactose or mannitol or an oily filler, e.g. olive, peanut or paraffin oil.

Aqueous suspensions can contain suspension agents, e.g. sodium carboxymethyl cellulose, methyl cellulose, hydroxypropyl cellulose, sodium alginate, polyvinyl pyrrolidon, traganth rubber or arabine; dispersant or wetting agents, e.g. polyoxyethylene stearate, heptadeca-ethylene-oxycatanol, polyoxyethylene sorbitol-monooleate, or lecithin; preservatives, e.g. methyl- or propylhydroxy-benzoate; taste modifiers; sweeteners, e.g. saccharose, sodium cyclamate, glucose, invert sugar syrup.

Oily suspensions may contain, for example, peanut, olive, sesame, coconut or paraffin oil and thickening agents, such as bees wax, high melting point wax or cetyl alcohol; also sweeteners, taste modifiers and antioxidants.

Powder and granulates dispersible in water may contain the compound according to the invention in a mixture with dispersing, wetting and suspension agents, e.g. those mentioned above as well as with sweeteners, taste modifiers and colorants.

Emulsions can, for example, contain olive, peanut or paraffin oil as well as emulsifying agents such as arabine, traganth rubber, phosphatides, sorbitan monooleate, polyoxyethylene sorbitan monooleate and sweeteners and taste modifiers.

Aqueous solutions can contain preservatives, e.g. methyl- or propylhydroxybenzoates, thickening agents; taste modifiers; sweeteners, e.g. saccharose, sodium cyclamate, glucose, invert sugar syrup as well as colorants.

For the parenteral application of the agent sterile injectable aqueous solutions, isotonic salt solutions or other solutions can be used.

### EXAMPLE 1

### (SP-4-2)-[bis(2-hydroxyethylamine)]diiodoplatinum(II)

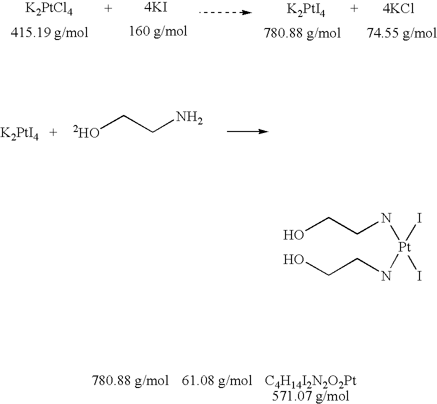

Initial Weights:

| | | |
|---|---|---|
| K₂PtCl₄ | 6 g | (14.45 mmol) |
| Kl | 12 g | (72.28 mmol) |
| Ethanol amine | 1.94 g | (31.76 mmol) |

6 g of K₂PtCl₄ are completely dissolved in 60 ml of H₂O tridest and mixed with 12 g of Kl. The solution becomes brown colored. It is stirred for 10 min at room temperature and the ethanol amine, dissolved in 10 ml of H₂O tridest., is added in 1 ml portions. A yellow, crystalline precipitate starts to settle out. The reaction is terminated after 3 hours of stirring and the supernatant solution is colored bright yellow. The precipitate is sucked out and washed twice with 5 ml of ice-cold water and twice with 3 ml of similarly ice-cold ethanol. The substance is dried over phosphorus pentoxide in a vacuum.

| | |
|---|---|
| Yield: | 6.839 g (82.88%) |
| Appearance: | Yellow crystalline precipitate |
| Decomposition point: | >140° C. |
| Elementary analysis: | Calculated for C₄H₁₄N₂O₂I₂Pt |

| | C | H | I | N | O | Pt |
|---|---|---|---|---|---|---|
| Calculated | 8.41 | 2.47 | 44.44 | 4.91 | 5.60 | 34.16 |
| Found | 8.42 | 2.20 | — | 4.80 | — | — |

IR spectrum (340-4400 cm⁻¹, KBr) characteristic bands (in cm⁻¹)

| | |
|---|---|
| 3500 | ν(O—H) |
| 3388-3125 | ν(N—H) |
| 2929, 2902 | ν(C—H) |
| 2877 | |

(SP-4-2)-dichloro[bis(2-hydroxyethylamine)]platinum(II)

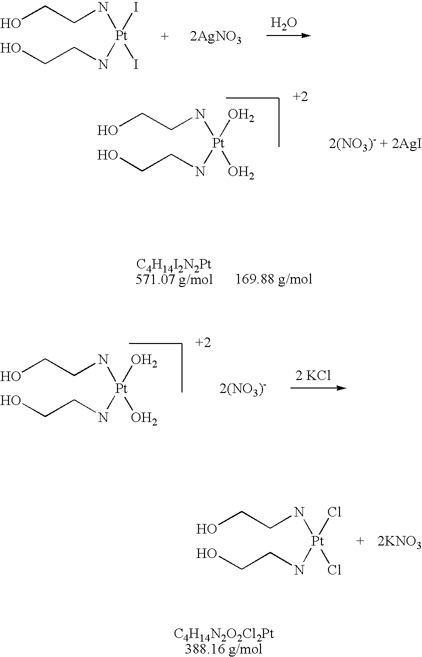

Initial Weights:

| | | |
|---|---|---|
| (SP-4-2)-[bis(2- | 2.9806 g | (5.22 mmol) |
| hydroxyethylamine)]diiodoplatinum(II) | | |
| AgNO₃ | 1.685 g | (9.92 mmol) |
| KCl | 1.6 g | (21.2 mmol) |

2.9806 g of (SP-4-2)-[bis(2-hydroxyethylamine)]diiodoplatinum(II) are suspended in 20 ml of H₂O tridest. and 5 ml of acetone. 1.685 g of AgNO₃ are added and stirred for 12 hours. The brown AgI is filtered off with a G4 filter crucible, rinsed twice with 5 ml of H₂O tridest. and centrifuged down to 10 ml. The filtrate is mixed with 1.6 g of KCl and frozen in liquid nitrogen. The substance is thawed out at 4° C., whereby pale yellow precipitate settles out. The compound is sucked off, washed twice with 5 ml of ice-cold water and dried in a vacuum over phosphorus pentoxide.

| | |
|---|---|
| Yield: | 1.29 g (63.66%) |
| Appearance: | Pale yellow solid |
| Decomposition point: | 145° C. |
| Elementary analysis: | Calculated for C₄Cl₂H₁₄N₂O₂Pt |

| | C | Cl | H | N | O | Pt |
|---|---|---|---|---|---|---|
| Calculated | 12.38 | 18.27 | 3.64 | 7.22 | 8.24 | 50.26 |
| Found | 12.21 | — | 3.37 | 6.92 | — | — |

IR spectrum (340-4400 cm⁻¹)

| | |
|---|---|
| 3468 | ν(O—H) |
| 3254, 3100 | ν(N—H) |
| 2913-2789 | ν(C—H) |
| 1569 | δ(N—H) |

NMR Spectrum:

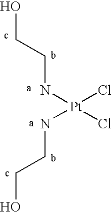

¹H NMR (H₂O/D₂O, 9:1): δ=4.07 [m, 4H, Ha], 3.78 [t, 2H, Hb], 3.70 [t, 2H, Hb], 2.96 [m, 2H, Hc], 1.81 [m, 2H, Hd]

(SP-4-2)-[bis(2-aminoethanolato)-κ²N,O]platinum(II)

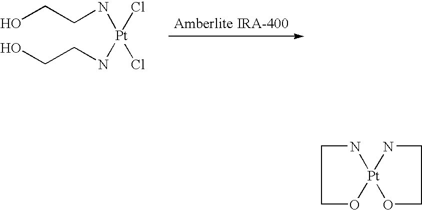

| C₄Cl₂H₁₄N₂O₂Pt | C₄H₁₂N₂O₂Pt |
|---|---|
| 388.16 g/mol | 315.24 g/mol |

Initial Weights:

(SP-4-2)-dichloro[bis(2-hydroxyethylamine)]platinum(II) 0.35 g (0.9 mmol)

30 ml of Amberlite IRA-400 (ion exchanger), in 2 M NaOH stirred for 30 min and washed three times with 20 ml of H₂O.

0.35 g (SP-4-2)-dichloro[bis(2-hydroxyethylamine)]platinum(II) are suspended in 10 ml of H₂O tridest. and stirred for 24 hours with 30 ml of conditioned Amberlite IRA-400 (Cl⁻-form). The ion exchanger is filtered off with a blue-band filter and the filtrate is concentrated to 1-2 ml on the rotavapor. The colorless solution is slowly concentrated in the exsiccator over phosphorus pentoxide until transparently clear crystals settle out. The crystals are dried on a filter paper.

| | |
|---|---|
| Yield: | 0.136 g (47.86%) |
| Appearance: | Colorless crystals |
| Decomposition point: | 137° C. |
| Elementary analysis: | Calculated for C₄H₁₂N₂O₂Pt |

| | C | H | N | O | Pt |
|---|---|---|---|---|---|
| Calculated | 15.24 | 3.84 | 8.89 | 10.15 | 61.89 |
| Found | 15.08 | 3.60 | 8.61 | — | — |

IR Spectrum (400-4400 cm⁻¹, KBr) characteristic bands (in cm⁻¹)

| | |
|---|---|
| 3210, 3035 | ν(N—H) |
| 2815 | ν(C—H) |
| 1560 | δ(N—H) |
| 545 | ν(Pt—O) |

NMR Spectra:

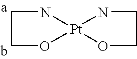

¹H NMR (H₂O/D₂O, 9:1): δ=2.96 [t, (³J(H^{a}, H^{b})=5.52 Hz) 4H, H^{b}], 2.24 [t, (³J(H^{b}, H^{a})=5.52 Hz), 4H, H^{a}) ¹³C-NMR (D₂O): δ=69.31 (2C, C^{b}), 50.80 (2C, C^{a}) ¹⁵N-NMR (D₂O): δ=−44.05 (2N)

### EXAMPLE 2

### (OC-6-22)-tetrachloro[bis(2-hydroxyethylamine)]platinum(IV)

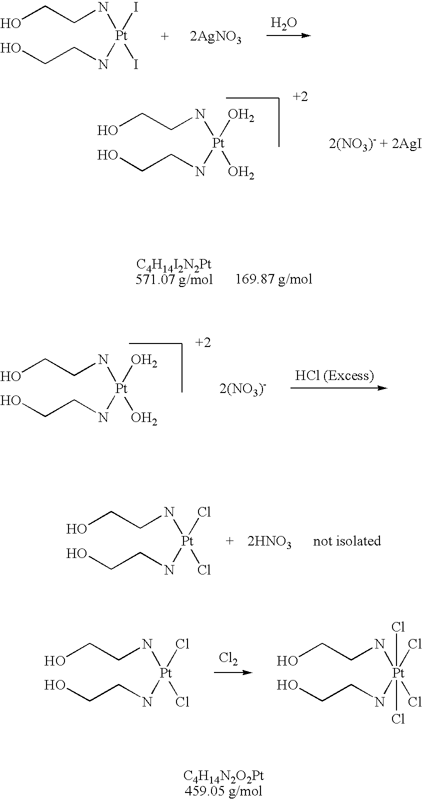

Initial Weights:

| | | | |
|---|---|---|---|
| (SP-4-2)-[bis(2- | 2.5181 | g | (4.41 mmol) |
| hydroxyethylamine)]diiodoplatinum(II) | | | |
| AgNO₃ | 1.4232 | g | (8.38 mmol) |
| HCl, fuming | 6.56 | ml | (81.12 mmol) |

2.5181 g of (SP-4-2)-[bis(2-hydroxyethylamine)]diiodoplatinum(II) are suspended in 50 ml of H₂O tridest. and mixed with 1.4232 g AgNO₃. This is stirred for 12 hours and then the brown AgI is filtered via a G4 glass filter crucible. The filtrate is mixed with 6.56 ml of fuming HCl, whereby the solution color changes from bright yellow to dark yellow. Chlorine gas is then passed through for 200 min, whereby the solution becomes slightly turbid after 1.5 hours. It is then concentrated down to 20 ml and stored overnight in a refrigerator. The solid material is filtered off and washed with a little H₂O and ethanol. It is then dried in a vacuum over P₄O₁₀.

| | |
|---|---|
| Yield: | 1.4241 g (70.35%) |
| Appearance: | Yellow solid |
| Decomposition point: | 160° C. |
| Elementary analysis: | Calculated for C₄H₁₄Cl₄N₂O₂Pt |

| | C | H | Cl | N | O | Pt |
|---|---|---|---|---|---|---|
| Calculated | 10.47 | 3.47 | 30.89 | 6.10 | 6.97 | 42.50 |
| Found | 10.19 | 3.08 | — | 5.77 | — | — |

IR spectrum (340-4400 cm⁻¹, KBr) characteristic bands (in cm⁻¹)

| | |
|---|---|
| 3501 | ν(O—H) |
| 3196, 1620 | ν(N—H) |
| 3010, 2962 | ν(C—H) |

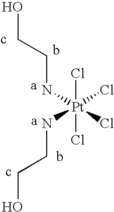

NMR spectra: ¹H NMR (H₂O/D₂O, 9:1): δ=6.36 [m, 4H, H^{a}], 3.70 [t, (³J(Hc,Hb)=5.23 Hz), 4H,Hc], 2.98 [m, 4H,Hb]. ¹⁵N NMR (H₂O/D₂O, 9:1): δ=−21.78 [¹J(N,Pt=228 Hz), ²J(H,Pt=60 Hz), Na]. ¹³C NMR:(H₂O/D₂O, 9:1): δ=59.4 (Cc), 48.2 (Cb).
(OC-6-31)-trichloro(2-aminoethanolato-κN,O)(2-hydroxyethylamine)-platinum(IV)

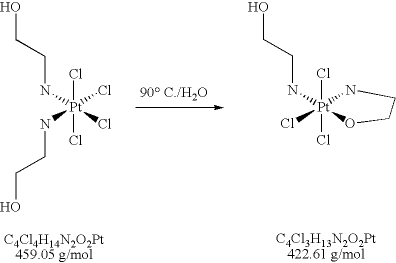

Initial Weights:

(OC-6-22)-tetrachloro[bis(2-hydroxyethylamine)]platinum(IV): 0.3 g (0.65 mmol)

0.3 g of (OC-6-22)-tetrachloro[bis(2-hydroxyethylamine)platinum(IV) is dissolved in 10 ml of H₂O tridest. and heated at 90° C. for 1.5 hours. Heating is then terminated and after 10 min orange crystals start to settle out. These are filtered off with a G4 filter crucible and washed twice with cold water and dried in a vacuum over P₄O₁₀.

| | |
|---|---|
| Yield: | 0.143 g (51.77%) |
| Appearance: | Orange, needle-shaped crystals |
| Decomposition point: | 174° C. |
| Elementary analysis: | Calculated for C₄Cl₃H₁₃N₂O₂Pt |

| | C | Cl | H | N | O | Pt |
|---|---|---|---|---|---|---|
| Calculated | 11.37 | 25.17 | 3.10 | 6.63 | 7.57 | 46.16 |
| Found | 11.42 | 25.12 | 2.92 | 6.39 | — | 45.66 |

IR spectrum (400-4400 cm⁻¹, KBr) characteristic bands (in cm⁻¹)

| | |
|---|---|
| 3501 | ν(O—H) |
| 3200 | ν(N—H) |
| 2900-2800 | ν(C—H) |
| 1547 | δ(N—H) |
| 572 | ν(Pt—O) |

NMR spectrum:

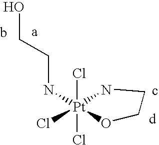

¹H NMR (H₂O/D₂O, 9:1): δ=3.67 [t, (³J(H^{b},H^{a})=5.3 Hz), 2H, H^{b}], 3.20 [t, (³J(H^{d}, H^{c})=5.77 Hz), 2H, H^{d}], 2.97 [t, (³J(H^{a}, H^{b})=5.48 Hz) 2H, H^{a}], 2.62 [t, (³J(H^{c}, H^{d})=5.58 Hz), 2H, H^{c}]
(OC-6-13)-dichloro[bis-(2-ethanolato)-κ²N,O)]platinum(IV)

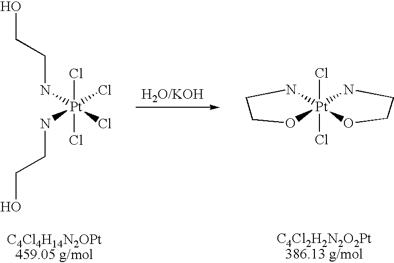

Initial Weights:

| | | |
|---|---|---|
| (OC-6-22)-tetrachloro[bis(2- | 0.1204 | g |
| hydroxyethylamine)]platinum(IV): | | |
| KOH: | 0.15 | g |

0.1204 g of (OC-6-22)-tetrachloro[bis(2-hydroxyethylamine)]platinum(IV) are dissolved in 5 ml of H₂O tridest. and mixed with 0.15 g of KOH. The solution is left to stand for 3 days and a pale yellow, needle-shaped precipitate starts to settle out. The precipitate is filtered off and washed with 3×5 ml of H₂O and dried in a vacuum over P₄O₁₀.

| | |
|---|---|
| Yield: | 0.0443 g (36.79%) |
| Appearance: | Pale yellow, needle-shaped solid |
| Decomposition point: | 185° C. |
| Elementary analysis: | Calculated for C₄Cl₂H₁₂N₂O₂Pt |

| | C | Cl | H | N | O | Pt |
|---|---|---|---|---|---|---|
| Calculated | 12.44 | 18.36 | 3.13 | 7.25 | 8.29 | 42.50 |
| Found | 12.59 | — | 2.95 | 7.01 | — | — |

IR spectrum (400-4400 cm⁻¹, KBr) characteristic bands (in cm⁻¹)

| | |
|---|---|
| 3190, 3100 | ν(N—H) |
| 2847 | ν(C—H) |
| 1596 | δ(N—H) |

NMR spectrum:

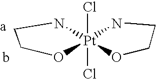

¹H NMR (H₂O/D₂O, 9:1): δ=3.14 [m, 4H, H^{a}], 2.62 [m, 4H, H^{b}]

### EXAMPLE 3

### (SP-4-2)-[bis(2-aminobutanol)]diiodoplatinum(II)

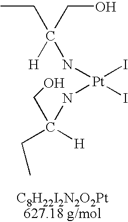

Initial Weights:

| | | |
|---|---|---|
| K₂PtCl₄: | 5 g | (12.04 mmol) |
| Kl: | 10 g | (60.24 mmol) |
| 2-aminobutanol: | 2.36 g | (26.47 mmol) |

5 g of K₂PtCl₄ are completely dissolved in 50 ml of H₂O tridest. and mixed with 10 g of KI. The solution becomes brown colored and is stirred for 20 min. 2.36 g of 2-aminobutanol are dissolved in 20 ml of water and added in 1 ml portions. The brown solution becomes brighter and after 10 min a yellow solid starts to settle out. To complete the precipitation, stirring continues for another 12 hours and then the precipitate is filtered off with a G4 filter crucible. The compound is washed twice with water and dried over P₄O₁₀.

| | |
|---|---|
| Yield: | 6.848 g (90.7%) |
| Appearance: | Yellow solid |
| Decomposition point: | 141° C. |
| Elementary analysis: | Calculated for C₈H₂₂I₂N₂O₂Pt |

| | C | H | I | N | O | Pt |
|---|---|---|---|---|---|---|
| Calculated | 15.32 | 3.54 | 40.47 | 4.47 | 5.10 | 31.11 |
| Found | 15.02 | 3.30 | — | 4.25 | — | 31.05 |

IR spectrum (400-4400 cm⁻¹, KBr) characteristic bands (in cm⁻¹)

| | |
|---|---|
| 3483 | ν(O—H) |
| 3255, 3197 | ν(N—H) |
| 2960-2871 | ν(C—H) |
| 1563 | δ(N—H) |

(SP-4-2)-[bis(2-aminobutanol)]dichloroplatinum(II)

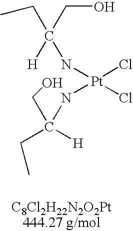

Initial Weights:

| | | |
|---|---|---|
| (SP-4-2)-[bis(2- | 4.0000 g | (6.37 mmol) |
| aminobutanol)]diiodoplatinum(II): | | |
| AgNO₃: | 2.0802 g | (12.24 mmol) |
| KCl: | 1.05 g | (14.08 mmol) |

4 g of (SP-4-2)-[bis(2-aminobutanol)]diiodoplatinum(II) are suspended in 20 ml of water and 5 ml of acetone and 2.0802 g AgNO₃ are added. After 24 hours of stirring, brown AgI is sucked off. The bright yellow filtrate is mixed with 1.05 g KCl and after 5 min yellow, needle-shaped crystals begin to separate out. The product is stored for 3 hours at 4° C. The crystalline mass is filtered off and washed twice with 5 ml of water and dried over phosphorus pentoxide.

| | |
|---|---|
| Yield: | 1.3898 g (46.9%) |
| Appearance: | Yellow, crystalline solid |
| Decomposition point: | 157° C. |
| Elementary analysis: | Calculated for C₈Cl₂H₂₂N₂O₂Pt |

| | C | Cl | H | N | O | Pt |
|---|---|---|---|---|---|---|
| Calculated | 21.63 | 15.96 | 4.99 | 6.31 | 7.20 | 43.91 |
| Found | 21.72 | 15.81 | 4.79 | 6.25 | — | 43.86 |

IR spectrum (400-4400 cm⁻¹, KBr) characteristic bands (in cm⁻¹)

| | |
|---|---|
| 3467 | ν(O—H) |
| 3230, 3123 | ν(N—H) |
| 2962 | ν(C—H) |
| 1572 | δ(N—H) |

(SP-4-2)-[bis(2-amino-1-butanolato)-κ²N,O]platinum(II)

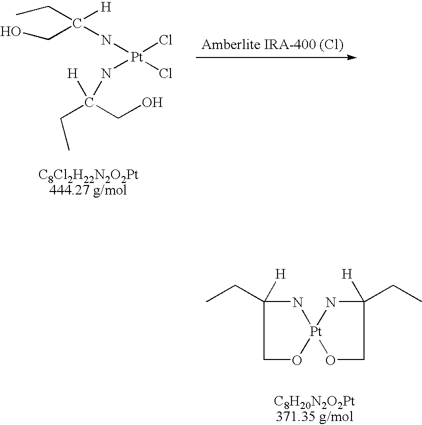

Initial Weights:

(SP-4-2)-[bis(2-aminobutanol)]dichloroplatinum(II): 0.3001 g (0.67 mmol)

0.3 g of (SP-4-2)-dichloro[bis(-2-aminobutanol)]platinum(II) is suspended in 10 ml of H₂O tridest. and mixed with 30 ml of Amberlite IRA-400 (Cl⁻ form). After the suspension has been shaken for 24 hours, the ion exchange resin is filtered off with a blue-band filter and washed twice with 10 ml of water. The colorless solution is concentrated by rotary evaporation to 1-2 ml and concentrated over phosphorus pentoxide until the crystalline solid precipitates. This is collected on a filter paper and dried.

| | |
|---|---|
| Yield: | 0.178 g (70.96%) |
| Appearance: | Yellow, needle-shaped solid |
| Decomposition point: | 199° C. |
| Elementary analysis: | Calculated for C₄H₁₂N₂O₂Pt × 2H₂O |

| | C | H | N | O | Pt |
|---|---|---|---|---|---|
| Calculated | 23.59 | 5.94 | 6.88 | 15.71 | 49.89 |
| Found | 23.86 | 5.54 | 6.60 | — | 51.47 |

IR spectrum (400-4400 cm⁻¹, KBr) characteristic bands (in cm⁻¹)

| | |
|---|---|
| 581 | ν(Pt—O) |
| 3066, 3003 | ν(N—H) |
| 2916 | ν(C—H) |
| 1636 | δ(N—H) |

### EXAMPLE 4

### (SP-4-2)-[bis-R-(−)-2-aminobutanol)]diiodoplatinum(II)

| | | |
|---|---|---|
| K₂PtCl₄: | 3.6212 g | (8.72 mmol) |
| KI: | 7.5 g | (45.18 mmol) |
| R-(−)-2-aminobutanol: | 1.45 g | (16.27 mmol) |

3.6121 g of K₂PtCl₄ are dissolved in 35 ml of H₂O tridest. and mixed with 7.5 g of KI. 1.45 g of R-(−)-2-aminobutanol are dissolved in 10 ml of water tridest. and added drop by drop. A yellow precipitate starts to separate out from a red solution. After 5 hours of stirring, the precipitate can be sucked off. It is washed twice with 10 ml of water and each time 1× with 5 ml of ethanol and 5 ml of diethyl ether. The substance is dried in a vacuum over P₄O₁₀.

| | |
|---|---|
| Yield: | 2.7216 g (49.75%) |
| Appearance: | Yellow solid |
| Decomposition point: | 127° C. |
| Elementary analysis: | Calculated for C₈H₂₂I₂N₂O₂Pt |

| | C | H | I | N | O | Pt |
|---|---|---|---|---|---|---|
| Calculated | 15.32 | 3.54 | 40.47 | 4.47 | 5.10 | 31.11 |
| Found | 15.36 | 3.27 | — | 4.29 | — | — |

IR spectrum (400-4400 cm⁻¹, KBr) characteristic bands (in cm⁻¹)

| | |
|---|---|
| 3482 | ν(O—H) |
| 3199, 3110 | ν(N—H) |
| 2960-2872 | ν(C—H) |
| 1563 | δ(N—H) |

(SP-4-2)-dichloro[bis-(R-(−)-2-aminobutanol)]platinum(II)

Initial Weights:

| | | |
|---|---|---|
| (SP-4-2)-diiodo[bis(R- | 1.5000 | g (3.61 mmol) |
| (-)-2-aminobutanol)]platinum(II): | | |
| AgNO₃: | 0.7720 | g (4.54 mmol) |
| HCl: | 1.4 | ml (16.38 mmol) |

1.5 g (SP-4-2)-[bis(R-(−)-2-aminobutanol)]diiodoplatinum(II) are suspended in 20 ml of H₂O tridest. and mixed with 0.772 g AgNO₃. The suspension is shaken for 12 hours and then the precipitated, brown AgI is filtered off. The filtrate is concentrated to 10 ml and 1.4 ml of HCl added to it. After 5 min yellow, needle-shaped crystals begin to separate out. The substance is stored at 4° C. for 3 hours. The crystals are sucked off and washed three times with 5 ml of ice-cold water.

| | |
|---|---|
| Yield: | 0.8212 g (77.3%) |
| Appearance: | Yellow, needle-shaped crystals |
| Decomposition point: | 160° C. |
| Elementary analysis: | Calculated for C₈Cl₂H₂₂N₂O₂Pt |

| | C | Cl | H | N | O | Pt |
|---|---|---|---|---|---|---|
| Calculated | 21.63 | 15.96 | 4.99 | 6.31 | 7.20 | 43.91 |
| Found | 21.56 | — | 4.87 | 6.17 | — | — |

IR spectrum (340-4400 cm⁻¹, KBr) characteristic bands (in cm⁻¹)

| | |
|---|---|
| 3470 | ν(OH) |
| 3234-3124 | ν(NH) |
| 2962-2875 | ν(CH) |
| 1570 | δ(NH) |

(SP-4-2)-[bis-(R-(−)-2-amino-1-butanolato)-κ²N,O]platinum(II)

Initial Weights:

(SP-4-2)-[bis(R-(−)-2-aminobutanol)]dichloroplatinum(II): 0.3 g (0.67 mmol)

0.3 g of (SP-4-2)-[bis(R-(−)-2-aminobutanol)]dichloroplatinum(II) are suspended in 10 ml of H₂O tridest. and mixed with 30 ml of Amberlite IRA-400 (Cl⁻ form). After the suspension has been shaken for 24 hours, the ion exchange resin is filtered off with a blue-band filter and washed twice with 10 ml of water. The colorless solution is concentrated by rotary evaporation to 1 ml and then concentrated over P₄O₁₀ until colorless crystals precipitate. These are collected on a filter paper and dried.

| | |
|---|---|
| Yield: | 0.149 g (59.4%) |
| Appearance: | Colorless, transparently clear crystals |
| Decomposition point: | 156° C. |

The elementary analysis of the substance did not correspond to the calculated values. The compound was characterized with crystallographic methods.

IR spectrum (400-4400 cm⁻¹, KBr) characteristic bands (in cm⁻¹)

| | |
|---|---|
| 3234, 3124 | ν(N—H) |
| 2962-2875 | ν(C—H) |
| 1570 | δ(N—H) |

NMR spectrum: ¹H NMR (H₂OD₂0, 9:1): δ=3.06 [m, 2H, H^{b}], 2.94 [m, 2H, H^{b}], 2.54 [m, 2H, H^{a}], 1.59 [mm (³J(H^{d}, H^{a}, H^{c})=7.33 Hz) 2H, H^{c}], 1.42 [m, (³J(H^{d}, H^{a}, H^{c})=7.1 Hz), 2H, H^{c}], 0.86 [t, (³J(H^{c}, H^{d})=7.58), 4H, H^{d}]

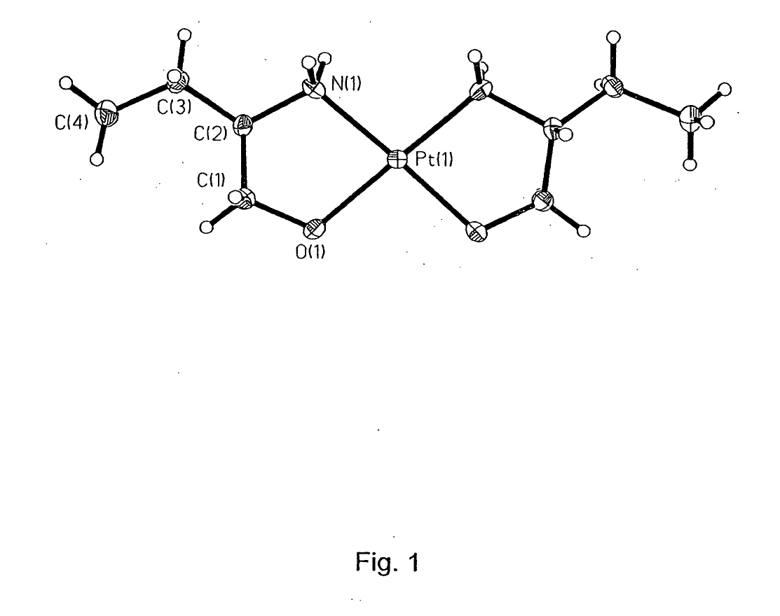

The X-ray structural analysis of the manufactured compound can be taken from FIG. 1.

### EXAMPLE 5

### (SP-4-2)-bis[S-(+)-2-amino-1-butanolato)diiodoplatinum(II)

Initial Weights:

| | | |
|---|---|---|
| K₂PtCl₄: | 3 g | (7.23 mmol) |
| KI: | 6 g | (36.14 mmol) |
| S-(+)-2-amino-1-butanol: | 1.42 g | (15.92 mmol) |

3 g of K₂PtCl₄ are dissolved in 30 ml of H₂O tridest. and mixed with 6 g of Kl. The solution becomes brown and is stirred for 20 min. Then 1.41 g S-(+)-2-amino-1-butanol in 15 ml of H₂O is added to the solution in portions. The solution is stirred for 12 hours so that the supernatant solution no longer exhibits any red coloration. The precipitation is filtered off and washed with water twice. The substance is dried in a vacuum over phosphorus pentoxide.

| | |
|---|---|
| Yield: | 3.4362 g (75.8%) |
| Appearance: | Yellow solid |
| Decomposition point: | 132° C. |
| Elementary analysis: | Calculated for C₈H₂₂I₂N₂O₂Pt |

| | C | H | I | N | O | Pt |
|---|---|---|---|---|---|---|
| Calculated | 15.32 | 3.54 | 40.47 | 4.47 | 5.10 | 31.11 |
| Found | 15.29 | 3.38 | — | 4.92 | — | 30.92 |

IR spectrum (400-4400 cm⁻¹, KBr) characteristic bands (in cm⁻¹)

| | |
|---|---|
| 3465 | ν(O—H) |
| 3219, 3113 | ν(N—H) |
| 2959-2871 | ν(C—H) |
| 1565 | δ(N—H) |

(SP-4-2)-dichloro-[bis(S-(+)-2-aminobutanol)]platinum(II)

Initial Weights:

| | | |
|---|---|---|
| (SP-4-2)-[bis-(S-(+)-2- | 1.8 g | (2.87 mmol) |
| aminobutanol)]diiodoplatinum(II): | | |
| AgNO₃: | 0.9361 g | (5.51 mmol) |
| KCl: | 0.53 g | (7.11 mmol) |

1.8 g (SP-4-2)-[bis(S-(+)-2-aminobutanol)]diiodoplatinum(II) are suspended in 20 ml of H₂O tridest. and mixed with 0.9361 g of AgNO₃. The suspension is stirred for 12 hours and the precipitated, brown AgI is filtered off. The filtrate is mixed with 0.53 g KCl and the color changes from bright yellow to intensive yellow. After 5 min yellow, needle-shaped crystals begin to separate out. After 3 hours of storage at 4° C., the crystalline solid material is filtered off with a G4 filter crucible. The substance is washed with water twice and dried over phosphorus pentoxide.

| | |
|---|---|
| Yield: | 0.55 g (41.2%) |
| Appearance: | Yellow, crystalline solid |
| Decomposition point: | 145° C. |
| Elementary analysis: | Calculated for C₈Cl₂H₂₂N₂O₂Pt × |
| | 0.1 C₈ClH₂₀N₂O₂Pt |

| | C | Cl | H | N | O | Pt |
|---|---|---|---|---|---|---|
| Calculated | 21.79 | 15.35 | 5.01 | 6.35 | 7.26 | 44.24 |
| Found | 21.50 | 15.35 | 4.84 | 6.84 | — | 43.96 |

IR spectrum (400-4400 cm⁻¹, KBr) characteristic bands (in cm⁻¹)

| | |
|---|---|
| 3471 | ν(O—H) |
| 3209, 3124 | ν(N—H) |
| 2964-2877 | ν(C—H) |
| 1569 | δ(N—H) |

NMR spectrum: ¹H NMR (H₂OD₂O, 9/1): δ=4.36 [m, 4H, H^{a}], 3.60-3.22 [m, 4H, H^{c}], 2.32 [m, 4H, H^{b}], 1.30-1.08[m, 4H, H^{d}], 0.43 [t, (³J(H^{d})=7.58), 6H, H^{e}]

(SP-4-2)-[bis(2-S-(+)-amino-1-butanolato)]-κ²N,O platinum(II)

Initial Weights:

(SP-4-2)-dichloro-(bis-[S-(+)-amino-1-butanol])platinum(II): 0.1562 g (0.35 mmol) 20 ml of Cond. Amberlite IRA-400 (Cl⁻ Form)

0.1562 g of (SP-4-2)-dichloro-(bis-[S-(+)-amino-1-butanol])platinum(II) are suspended in 10 ml of H₂O tridest. and shaken with 20 ml of conditioned Amberlite IRA-400 (Cl⁻ form) for 2 days. The supernatant solution is colorless. The ion exchanger is filtered off with a blue-band filter and washed twice. The filtrate is concentrated down to 1-2 ml and stored over P₄O₁₀ until colorless crystals precipitate. The crystals are dried on a filter paper.

| | |
|---|---|
| Yield: | 0.07 g (53.6%) |
| Appearance: | Colorless, transparently clear crystals |
| Decomposition point: | 198° C. |

The elementary analysis of the substance did not correspond to the calculated values. The compound was characterized by crystallographic methods.

IR spectrum (400-4400 cm⁻¹, KBr) characteristic bands (in cm⁻¹)

| | |
|---|---|
| 3200, 2965, | ν(N—H) |
| 2950-2800 | ν(C—H) |
| 1550 | δ(N—H) |

NMR spectrum: ¹H NMR (H₂O/D₂O, 9:1): δ=3.00-2.88 [m, 4H, H^{b}], 2.48 [m, 2H, H^{a}], 1.52-1.36 [m, 4H, H^{c}], 0.80 [t, (³J(H^{c}, H^{d})=7.34), 6H, H^{d}]

### EXAMPLE 6

### Synthesis of bis(2-ethanolatoamine-κ²N,O)dihydroxoplatinum(IV) via cis-bis(2-ethanolatoamine-κ²N,O)platinum(II)

20 ml of Amberlite IRA 400 (strong basic ion exchanger) is stirred with 60 ml of a 2M NaOH for one hour and then washed 3 times with 20 ml of water.

300 mg of the compound cis-dichloro-bis(2-ethanolamine)platinum(II) are dissolved in 20 ml of water and added to the ion exchanger. The mixture is stirred overnight, filtered off and concentrated. This can be simplified by the addition of ethanol.

Colorless crystals or a white powder of the substance bis(2-ethanolatoamine-κ²N,O)platinum(II) were obtained which still contained 2 equivalents of water.

Analysis:

1. Elementary Analysis,

| | | | |
|---|---|---|---|
| calculated (C₄H₁₂N₂O₂Pt · 2H₂O): | C, 13.68; | H, 4.59; | N, 7.98 |
| found: | C, 13.86; | H, 4.26; | N, 7.69 |

2. NMR ¹H NMR (H₂O/D₂O): 2.25 (m, 2H, CH₂—NH₂), 2.96 (m, 2H, CH₂—O), ¹³C NMR (H₂O/D₂O): 50.9 (2C, CH₂—NH₂), 69.3 (2C, CH₂—O)

The corresponding platinum(IV) derivative, bis(2-ethanolatoamine-κ²N,O)dihydroxoplatinum(IV) is synthesised by oxidation in an aqueous alkali solution after the addition of 30% H₂O₂ and after stirring overnight at room temperature.

### EXAMPLE 7

### Synthesis of bis(2-amino-1-propanolato-κ²N,O)platinum(II) and bis(2-amino-1-propanolato-κ²N,O)dihydroxoplatinum(IV)

20 ml of Amberlite IRA 400 (strong basic ion exchanger) is stirred for one hour with 60 ml of a 2M NaOH and then washed 3 times with 20 ml of water.

325 mg of the compound cis-dichloro-bis(2-amino-1-propanol)platinum(II) are dissolved in 20 ml of water and added to the ion exchanger. The mixture is stirred overnight, filtered off and concentrated. This can be simplified by the addition of ethanol.

Colorless crystals or a white powder of the substance bis(2-amino-1-propanolato-κ²N,O)platinum(II) are obtained. The corresponding platinum(IV) derivative is synthesized by oxidation in an aqueous alkali solution after adding 30% H₂O₂ and after stirring overnight at room temperature.

Cytostatic Effect

### EXAMPLE 8

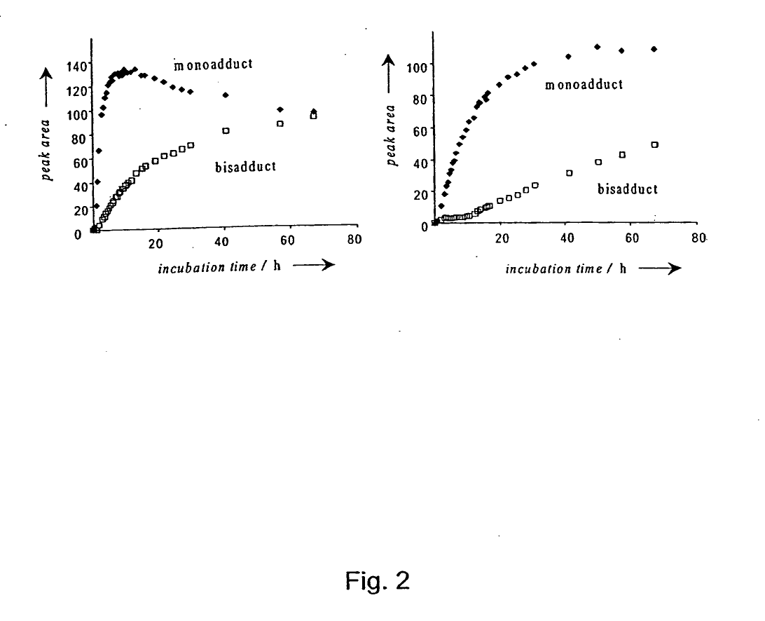

The reactivity of the compound cis-dichloro-bis(2-ethanolamine)platinum(II) was examined under physiological and patho-physiological conditions (FIG. 2). As the pharmacodynamic termination point, the formation of adducts of the effective substance with GMP as DNA constituent is generally accepted (Sartori, David A.; Miller, Bernhard; Bierbach, Ulrich; Farrell, N. “Modulation of the chemical and biological properties of trans platinum complexes: monofunctional platinum complexes containing one nucleobase as potential antiviral chemotypes” J. Biol. Inorg. Chem. 5(5): 575-583 (2000); Sullivan, Sharon T.; Ciccarese, Antonella; Fanizzi, Francesco P.; Marzilli, Luigi G. “Cisplatin-DNA Cross-link models with an Unusual Type of Chirality-Neutral Chelate Amine Carrier Ligand, N,N′-Dimethylpiperazine (Me2ppz): Me2ppzPt(guanosine monophosphate)2 Adducts That Exhibit Novel” Inorg. Chem. 39(4): 836-842 (2000); Choi, Sunhee; Mahalingaiah, Shruthi; Delaney, Sarah; Neale, Nathan R.; Masood, Syed “Substitution and Reduction of Platinum(IV) Complexes by a Nucleotide, Guanosine 5′-Monophosphate” Inorg. Chem. 38(8): 1800-1805 (1999); Chen, Yu; Guo, Zijian; Parkinson, John A.; Sadler, Peter J. “Kinetic control of reactions of a sterically hindered platinum picoline anticancer complex with guanosine 5′-monophosphate and glutathione” J. Chem. Soc., Dalton Trans.), 21: 3577-3586 (1998); Bloemink, Marieke J.; Perez, Janice M. J.; Heetebrij, Robert J.; Reedijk, Jan “The new anticancer drug [(2R)-aminomethylpyrrolidine](1,1-cyclobutanedicarboxylato)platinum(II) and its toxic S enantiomer do interact differently with nucleic acids” J. Biol. Inorg. Chem. 4(5): 554-567 (1999).

As can be seen from FIG. 2, the left diagram of FIG. 2 shows a rapid reaction with the biological target, whereas on the right, under physiological conditions, the reaction clearly runs slower. Selectivity is therefore present.

### EXAMPLE 9

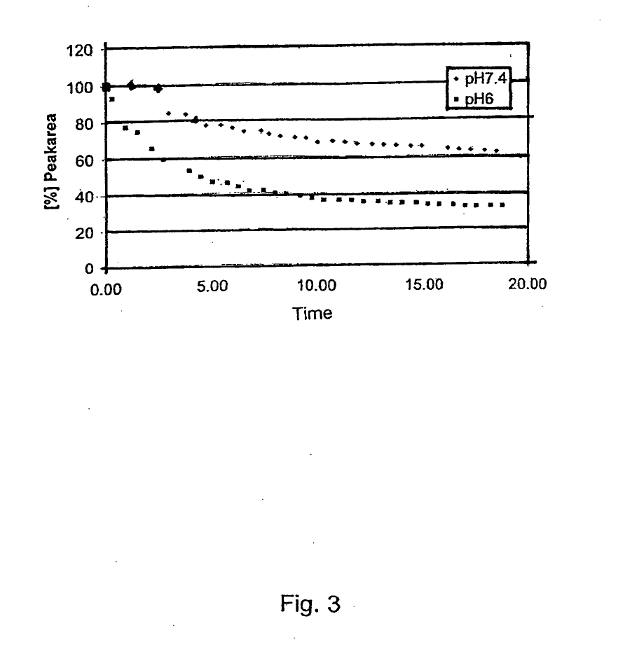

Similar to Example 8, the reactivity of the compound bis(2-ethanolatoamine-κ²N,O)platinum(II) (see Example 8 for the synthesis) was examined. In this respect, the proportion of the nucleotide not modified by the active substance was measured. FIG. 3 shows the significantly increased speed of this reaction observed under patho-physiological conditions (pH 6) and therefore a significant increase in the reactivity compared to tissue free of disease (pH 7.4).

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A platinum(II) complex selected from the group consisting of compounds of the general formulae I to IV and physiologically acceptable addition salts of them, 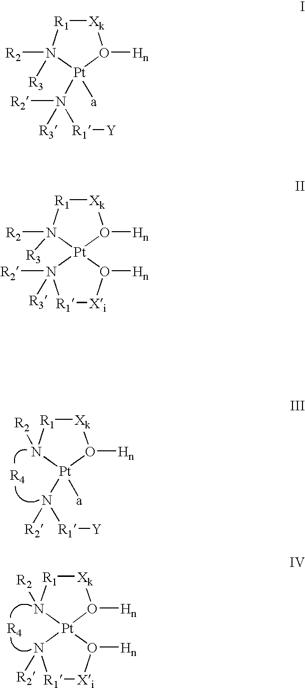
wherein the radicals
R¹ and R^{1′} are selected independently of one another from the group consisting of substituted or unsubstituted alkylene and alkenylene radicals, which can be substituted by halogen, alkyl, alkenyl, cycloalkyl, cycloalkenyl, aryl, hydroxy, carboxy, sulphate, phosphate and/or heterocyclic compounds,
R², R³, R^{2′}, R^{3′} are selected independently of one another from the group consisting of —(CH₂)ₘ—OH, —H, substituted or unsubstituted alkyl radicals, saturated and unsaturated cyclic radicals and heterocyclic compounds which can be substituted by halogen, hydroxy, carboxy, sulphate and/or phosphate,
m signifies a natural number from 2 to 5,
R⁴ is selected from the group consisting of substituted or unsubstituted alkylene, alkenylene, cycloalkylene and cycloalkenylene radicals, and aromatic and heterocyclic radicals,
X, X′=—S(O₂)—,
k, i=0 or 1,
Y=—OH, —SO₃H,
n=0 or 1, and
a is selected from the group consisting of halogenides, OH⁻, OH₂, carboxylate, sulphate and sulphonate
and mixtures of the compounds for use as prophylactic and/or therapeutic agent for the treatment of diseases.

2. A platinum(IV) complex with a single or double intramolecular cyclization selected from the group consisting of compounds of the general formulae Ia to IVa and physiologically acceptable addition salts of them, 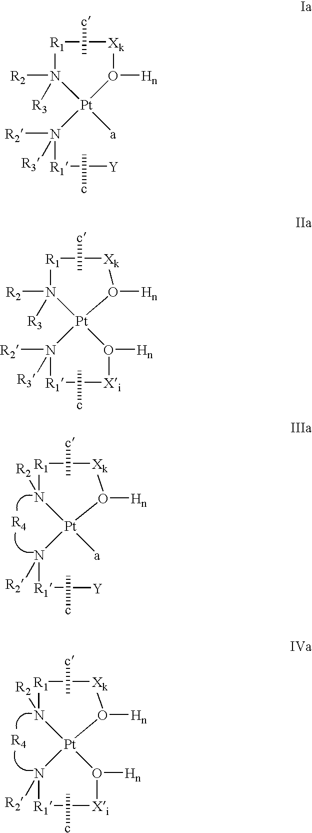
wherein the radicals
R¹ and R^{1′} are selected independently of one another from the group consisting of substituted or unsubstituted alkylene and alkenylene radicals, which can be substituted by halogen, alkyl, alkenyl, cycloalkyl, cycloalkenyl, aryl, hydroxy, carboxy, sulphate, phosphate and/or heterocyclic compounds,
R², R³, R^{2′}, R^{3′} are selected independently of one another from the group consisting of —(CH₂)ₘ—OH, —H, substituted or unsubstituted alkyl radicals, saturated and unsaturated cyclic radicals or heterocyclic compounds which can be substituted by halogen, hydroxy, carboxy, sulphate and/or phosphate,
m signifies a natural number from 2 to 5,
R⁴ is selected from the group consisting of substituted or unsubstituted alkylene, alkenylene, cycloalkylene and cycloalkenylene radicals, and aromatic and heterocyclic radicals,
X, X′=—S(O²)—,
k, i=0 or1,
Y=—OH, —SO₃H,
n=0or 1, and
a, c, c′ are selected independently of one another from the group consisting of halogenides, OH⁻, OH₂, carboxylate, sulphate and sulphonate
and mixtures of the compounds for use as prophylactic and/or therapeutic agent in the treatment of diseases.

3. The platinum(II) complex according to claim 1, wherein
R², R³, R^{2′}, R^{3′} are selected independently of one another from the group consisting of hydrogen, methyl and ethyl radicals.

4. The platinum(II) complex according to claim 1, wherein R¹ and R^{1′} are selected independently of one another from the group consisting of C₂₋₅-alkylene radicals and C₂₋₅-alkenylene radicals.

5. The platinum(II) complex according to claim 1, wherein R⁴ is ethylene.

6. The platinum(II) complex according to claim 1, wherein the radicals have the following significance:
R¹, R^{1′}=substituted or non-substituted alkylene,
k, i=0, and/or
Y=—OH.

7. The platinum(II) complex according to claim 1, wherein the radicals have the following significance:
R¹, R^{1′}=ethylene,
k, i=0,
Y=—OH, and/or
n=0.

8. Use of at least one platinum(II) complex according to claim 1, for the manufacture of a prophylactic and/or therapeutic agent for the treatment of tumor diseases.

9. A platinum(II) or platinum(IV) compound according to the general formula (V),
[Pt^{II}(NH₃)ₙ(A)ₙ(Z)₂₋ₙ] or [Pt^{IV}(NH₃)ₙ(A)₁(Z)₂₋ₙX₂] (V)
wherein
n is equal to 0 or 1,
A, X are selected independently of one another from the group consisting of halogenides, OH⁻, carboxylate, sulphate and sulphonate, and
Z is selected from the group consisting of hydroxyalkyl amine, hydroxyalkenyl amine, sulphoalkyl amine, sulphoalkenyl amine, which is substituted at at least one of the CH₂— or CH groups by a halogen, alkyl, alkenyl, cycloalkyl, cycloalkenyl, aryl, hydroxyl, carboxyl, sulphate, phosphate radical and/or a heterocyclic compound and additionally the amino nitrogen can be substituted with these radicals, wherein
for n equal to 0, the two radicals Z present in the molecule can be linked via a radical selected from the group consisting of substituted or unsubstituted alkylene, alkenylene, cycloalkylene and cycloalkenylene radicals and a heterocyclic compound, preferably ethane-1,2-diyl
and their physiologically acceptable addition salts.

10. A platinum(II) or platinum(IV) compound according to the general formula (VI),
[Pt^{II}(A)₁(Z)₂] or [Pt^{IV}(A)₁(Z)₂X₂] (VI)
wherein
A and X are selected independently of one another from the group consisting of halogenides, OH⁻, OH₂, carboxylate, sulphate and sulphonate, and
Z is selected from the group consisting of hydroxyalkyl amine, hydroxyalkenyl amine, carboxyalkyl amine, carboxyalkenyl amine, sulphoalkyl amine and sulphoalkenyl amine, which is substituted at at least one of the CH₂— or CH groups by a halogen, alkyl, alkenyl, cycloalkyl, cycloalkenyl, aryl, hydroxyl, carboxyl, sulphate, phosphate radical and/or a heterocyclic compound and additionally can be substituted at the amino nitrogen by these radicals, wherein
the two Z radicals present in the molecule can be linked via a radical selected from the group consisting of alkylene, alkenylene, cycloalkylene and cycloalkenylene and a heterocyclic compound, or substituted alkylene, alkenylene, cycloalkylene and cycloalkenylene
and their physiologically acceptable addition salts.

11. The compound according to claim 10, wherein two radicals Z present in the molecule are linked via ethylene.

12. The platinum(II) and/or platinum(IV) compound according to claim 9, for use as a prophylactic and/or therapeutic agent for the treatment of diseases.

13. Use of at least one platinum(II) and/or platinum(IV) compound according to claim 9, for the manufacture of a prophylactic and/or therapeutic agent for the treatment of tumor diseases.

14. Use of platinum(II) a complex of the general formulae Ib to IVb for the manufacture of medicaments for the therapy of tumor diseases, 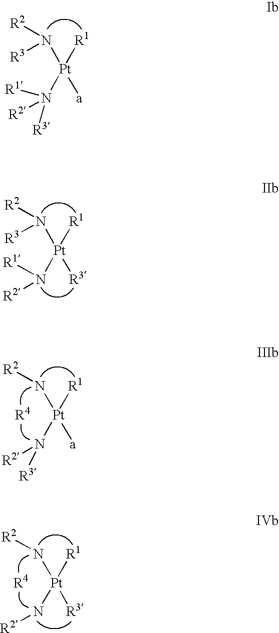
wherein the radicals
R¹ and R^{3′} originate from the group of hydroxyalkyls and hydroxyalkenyls and hydroxyalkyls and hydroxyalkenyls substituted with halogens, alkyls, cycloalkyls, heterocyclic compounds or functional groups such as hydroxy, carboxy, sulphate or phosphate, wherein the hydroxyalkyls and hydroxyalkenyls can be present protonated or deprotonated,
R², R³, R^{1′}, R^{2′}=—CH₂—CH₂—OH, —CH₂—CH₂—CH₂—OH, —CH₂—CH₂—CH₂—CH₂—OH, —CH₂—CH₂—CH₂—CH₂—CH₂—OH, —H, methyl, ethyl, saturated or unsaturated cyclic radicals, also heterocyclic compounds, as well as their halogen, hydroxy, carboxy, sulphate or phosphate derivatives,
R⁴=alkyl, alkylene, cycloalkyl, cycloalkene, heterocyclic radicals or substituted alkyls and alkylenes, cycloalkyl and cycloalkene, but preferably can be ethane-1,2-diyl,
and a belongs to the group of halogenides (fluorine, chlorine, bromine, iodine), OH⁻, OH₂, carboxylate, sulphate or sulphonate.

15. Use of a compound according to claim 14, wherein however R¹ and R^{3′}=carboxy alkyls or carboxy alkenyls as well as carboxy alkyls and carboxy alkenyls substituted by halogens, alkyls, cycloalkyls, heterocyclic compounds or functional groups such as hydroxy, carboxy, sulphate or phosphate and the carboxy alkyls or carboxy alkenyls can be present protonated or deprotonated.

16. Use of a compound according to claim 14, wherein however R¹ and R^{3′}=sulphoalkyls or sulphoalkenyls as well as sulphoalkyls and sulphoalkenyls substituted by halogens, alkyls, cycloalkyls, heterocyclic compounds or functional groups such as hydroxy, carboxy, sulphate or phosphate and the sulphoalkyls or sulphoalkenyls can be present protonated or deprotonated.

17. Use of a compound according to claim 14, wherein in particular
R¹ and R^{3′}=—CH₂—CH₂—OH, —CH₂—CH₂—CH₂—OH, —CH₂—CH₂—CH₂—CH₂—OH, —CH₂—CH₂—CH₂—CH₂—CH₂—OH, —CH₂—COOH, —CH₂—CH₂—COOH, —CH₂—CH₂—CH₂—COOH, —CH₂—CH₂—SO₃H, —CH₂—CH₂—CH₂—SO₃H, —CH₂—CH₂—CH₂—CH₂—SO₃H and —CH₂—CH₂CH₂—CH₂—CH₂—SO₃H and R¹ and R^{3′}=—CH₂—CH₂—OH, —CH₂—CH₂—CH₂—OH, —CH₂—CH₂—CH₂—CH₂—OH, —CH₂—CH₂—CH₂—CH₂—CH₂—OH, —CH₂—COOH, —CH₂—CH₂—COOH, —CH₂—CH₂—CH₂—COOH, —CH₂—CH₂—SO₃H, —CH₂—CH₂—CH₂—SO₃H, —CH₂—CH₂—CH₂—CH₂—SO₃H and —CH₂—CH₂—CH₂—CH₂—CH₂—SO₃H can be present protonated or deprotonated.

18. Use of a platinum(IV) complex with single or double intramolecular cyclizations of the general formulae Ic to IVc for the manufacture of medicaments for the therapy of tumor diseases, 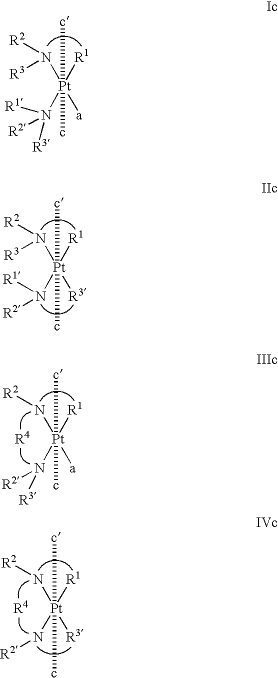
wherein the radicals
R¹ and R^{3′} originate from the group of hydroxyalkyls and hydroxyalkenyls, and hydroxyalkyls and hydroxyalkenyls substituted with halogens, alkyls, cycloalkyls, heterocyclic compounds or functional groups such as hydroxy, carboxy, sulphate or phosphate, wherein the hydroxyalkyls and hydroxyalkenyls can be present protonated or deprotonated,
R², R³, R^{1′}, R^{2′}=—CH₂—CH₂—OH, —CH₂—CH₂—CH₂—OH, —CH₂—CH₂—CH₂—CH₂—OH, —CH₂—CH₂—CH₂—CH₂—CH₂—OH, —H, methyl, ethyl, saturated or heterocyclic compounds, as well as their halogen, hydroxy, carboxy, sulphate or phosphate derivatives,
R⁴=alkyl, alkylene, cycloalkyl, cycloalkene, heterocyclic radicals or substituted alkyls and alkylenes, cycloalkyl and cycloalkene, but can preferably be ethane-1,2-diyl,
and a and c belong to the group of halogenides (fluorine, chlorine, bromine, iodine), OH⁻, OH₂, carboxylate, sulphate or sulphonate.

19. Use of a compound according to claim 18, wherein however R¹ and R^{3′}=carboxy alkyls or carboxy alkenyls as well as carboxy alkyls and carboxy alkenyls substituted with halogens, alkyls, cycloalkyls, heterocyclic compounds or functional groups such as hydroxy, carboxy, sulphate or phosphate and the carboxy alkyls or carboxy alkenyls can be present protonated or deprotonated.

20. Use of a compound according to claim 18, wherein however R¹ and R^{3′}=sulphoalkyls or sulphoalkenyls as well as sulphoalkyls and sulphoalkenyls substituted with halogens, alkyls, cycloalkyls, heterocyclic compounds or functional groups such as hydroxy, carboxy, sulphate or phosphate and the sulphoalkyls or sulphoalkenyls can be present protonated or deprotonated.

21. Use of a compound according to claim 18, in particular
R¹ and R^{3′}=—CH₂—CH₂—OH, —CH₂—CH₂—CH₂—OH, —CH₂—CH₂—CH₂—CH₂—OH, —CH₂—CH₂—CH₂—CH₂—CH₂—OH, —CH₂—COOH, —CH₂—CH₂—COOH, —CH₂—CH₂—CH₂—COOH, —CH₂—CH—SO₃H, —CH₂—CH₂—CH₂—SO₃H, —CH₂—CH₂—CH₂—CH₂—SO₃H and —CH₂—CH₂—CH₂—CH₂—CH₂—SO₃H and R¹ and R^{3′}=—CH₂—CH₂—OH, —CH₂—CH₂—CH₂—OH, —CH₂—CH₂—CH₂—CH₂—OH, —CH₂—CH₂—CH₂—CH₂—CH₂—OH, —CH₂—COOH, —CH₂—CH₂—COOH, —CH₂—CH₂—CH₂—COOH, —CH₂—CH₂—SO₃H, —CH₂—CH₂—CH₂—SO₃H, —CH₂—CH₂—CH₂—CH₂—SO₃H and —CH₂—CH₂—CH₂—CH₂—CH₂—SO₃H can be present protonated and deprotonated.

22. Use of a platinum compound according to claims 14, wherein however the radicals R¹ and R^{3′} preferably signify substituted and non-substituted hydroxyalkyls and wherein the hydroxyalkyls can be protonated or deprotonated.

23. Use of a platinum compound according to claim 14, wherein however the radicals R¹ and R^{3′} preferably signify 2-hydroxyethyl or deprotonated 2-hydroxyethyl, but especially preferably deprotonated 2-hydroxyethyl in the cyclized form.

24. A platinum(II) or platinum(IV) compound according to the general formula
[Pt^{II}(NH₃)ₙ(A)ₙ(Z)₂₋ₙ] or [Pt^{IV}(NH₃)ₙ(A)ₙ(Z)₂₋ₙX₂]
wherein
n is equal to 0 or 1,
A and X belong to the group of halogenides (fluorine, chlorine, bromine, iodine), OH⁻, OH₂, carboxylate, sulphate or sulphonate,
and Z,
provided n is equal to 0, the two radicals Z present in the molecule can be linked via a radical from the group of alkyls, alkylenes, cycloalkyls, cycloalkenyls, heterocyclic compounds or substituted alkyls and alkylenes, cycloalkyls and cycloalkenyls, but preferably ethane-1,2-diyl,
represents a hydroxyalkyl amine or hydroxyalkylene amine, carboxyalkyl amine or carboxyalkylene amine or sulphoalkyl amine or sulphoalkylene amine, which is derivatized on at least one of the CH₂ or CH groups with halogens, alkyls, cycloalkyls, heterocyclic compounds or functional groups such as hydroxy, carboxy, sulphate or phosphate and additionally can be derivatized on the amino nitrogen with these groups.

25. A platinum(II) or platinum(IV) compound according to the general formula
[Pt^{II}(A)₁(Z)₂] or [Pt^{IV}(A)₁(Z)₂X₂]
wherein
A and X belong to the group of halogenides (fluorine, chlorine, bromine, iodine), OH⁻, OH₂, carboxylate, sulphate or sulphonate, and
Z represents a hydroxyalkyl amine or hydroxyalkylene amine, carboxyalkyl amine or carboxyalkylene amine or sulphoalkyl amine or sulphoalkylene amine, which is derivatized on at least one of the CH₂ or CH groups with halogens, alkyls, cycloalkyls, heterocyclic compounds or functional groups such as hydroxy, carboxy, sulphate or phosphate and additionally can be derivatized on the amino nitrogen with these groups, wherein two radicals Z present in the molecule can be linked via a radical from the group of alkyls, alkylenes, cycloalkyls, cycloalkenyls, heterocyclic compounds or substituted alkyls and alkylenes, cycloalkyls and cycloalkenyls, but preferably ethane-1,2-diyl.

26. A platinum(II) or platinum(IV) compounds according to the general formula
[Pt^{II}(NH₃)ₙ(A)₂(Z)₂₋ₙ] or [Pt^{IV}(NH₃)ₙ(A)₂(Z)₂₋ₙX₂]
wherein
n is equal to 0 or 1,
A and X belong to the group of halogenides (fluorine, chlorine, bromine, iodine), OH⁻, OH₂, carboxylate, sulphate or sulphonate,
and Z,
provided n is equal to 0, the two radicals Z present in the molecule can be linked via a radical from the group of alkyls, alkylenes, cycloalkyls, cycloalkenyls, heterocyclic compounds or substituted alkyls and alkylenes, cycloalkyls, cycloalkenyls, but preferably ethane-1,2 diyl,
represents a hydroxyalkyl amine or hydroxyalkylene amine, carboxyalkyl amine or carboxyalkylene amine or sulphoalkyl amine or sulphoalkylene amine, which is derivatized on at least one of the CH₂ or CH groups with halogens, alkyls, cycloalkyls, heterocyclic compounds or functional groups such as hydroxy, carboxy, sulphate or phosphate and additionally can be derivatized on the amino nitrogen with these groups.

27. Use of a substance according to claim 24, for the manufacture of medicaments for the treatment of tumor diseases.

28. Use of a platinum(II) or platinum(IV) complex of the general formula V for the manufacture of medicaments for the therapy of tumor diseases 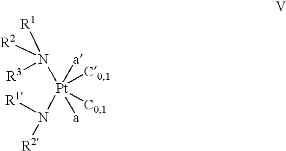
wherein the radicals R¹ and R^{3′} originate from the group of hydroxyalkyls and hydroxyalkenyls and hydroxyalkyls and hydroxyalkenyls substituted with halogens, alkyls, cycloalkyls, heterocyclic compounds or functional groups such as hydroxy, carboxy, sulphate or phosphate, wherein the hydroxyalkyls and hydroxyalkenyls can be present protonated or deprotonated,
R², R³, R^{1′}, R^{2′}=—CH₂—CH₂—OH, —CH₂—CH₂—CH₂—OH, —CH₂—CH₂—CH₂—CH₂—OH, —CH₂—CH₂—CH₂—CH₂—CH₂—OH, —H, methyl, ethyl, saturated or unsaturated cyclic radicals, also heterocyclic compounds, as well as their halogen, hydroxy, carboxy, sulphate or phosphate derivatives,
R⁴=alkyl, alkylene, cycloalkyl, cycloalkene, heterocyclic radicals or substituted alkyls and alkylenes, cycloalkyl and cycloalkene, but preferably ethane-1,2-diyl,
and a, a′ and c, c′ belong to the group of halogenides (fluorine, chlorine, bromine, iodine), OH⁻, OH₂, carboxylate, sulphate or sulphonate, wherein for platinum(II) compounds c is omitted (c0).

29. Use of a compound according to claim 28, wherein however R¹ and R^{3′}=carboxy alkyls or carboxy alkenyls as well as carboxy alkyls and carboxy alkenyls substituted with halogens, alkyls, cycloalkyls, heterocyclic compounds or functional groups such as hydroxy, carboxy, sulphate or phosphate and wherein the carboxy alkyls and carboxy alkenyls can be present protonated or deprotonated.

30. Use of a compound according to claim 28, wherein however R¹ and R^{3′}=sulphoalkyls or sulphoalkenyls as well as sulphoalkyls and sulphoalkenyls substituted by halogens, alkyls, cycloalkyls, heterocyclic compounds or functional groups such as hydroxy, carboxy, sulphate or phosphate and the sulphoalkyls or sulphoalkenyls can be present protonated or deprotonated.

31. Use of a compound according to claim 28, in particular
R¹ and R^{3′}=—CH₂—CH₂—OH, —CH₂—CH₂—CH₂—OH, —CH₂—CH₂—CH₂—CH₂—OH, —CH₂—CH₂—CH₂—CH₂—CH₂—OH, —CH₂—COOH, —CH₂—CH₂—COOH, —CH₂—CH₂—CH₂—COOH, —CH₂—CH₂—SO₃H, —CH₂—CH₂—CH₂—SO₃H, —CH₂—CH₂—CH₂—CH₂—SO₃H and —CH₂—CH₂CH₂—CH₂—CH₂—SO₃H and R¹ and R^{3′}=—CH₂—CH₂—OH, —CH₂—CH₂—CH₂—OH, —CH₂—CH₂—CH₂—CH₂—OH, —CH₂—CH₂—CH₂—CH₂—CH₂—OH, —CH₂—COOH, —CH₂—CH₂—COOH, —CH₂—CH₂—CH₂—COOH, —CH₂—CH₂—SO₃H, —CH₂—CH₂—CH₂—SO₃H, —CH₂—CH₂—CH₂—CH₂—SO₃H and —CH₂—CH₂—CH₂—CH₂—CH₂—SO₃H can be present protonated or deprotonated.

32. Use of a platinum compound according to claim 28, wherein however the radicals R¹ and R^{3′} preferably signify substituted and non-substituted hydroxyalkyls and wherein the hydroxyalkyls can be present protonated or deprotonated.

33. Use of a platinum compound according to claim 28, wherein however the radicals R¹ and R^{3′} preferably signify 2-hydroxyethyl or deprotonated 2-hydroxyethyl, but especially preferably deprotonated 2-hydroxyethyl in the cyclized form.

34. A medicament containing a compound of claim 14.
